SEQUENCE LISTING (66 sequences)

SEQ ID NO: 1 - DNA, 21 bp, synthetic construct
  source 1..21: mol_type = other DNA
agaccaatyytgtcacctctg

SEQ ID NO: 2 - DNA, 18 bp, synthetic construct
  source 1..18: mol_type = other DNA
cgggtccccattcccatt

SEQ ID NO: 3 - DNA, 22 bp, synthetic construct
  source 1..22: mol_type = other DNA
ggtttgagatattccccaarac

SEQ ID NO: 4 - DNA, 24 bp, synthetic construct
  source 1..24: mol_type = other DNA
aggatttgctgagctttgggtatg

SEQ ID NO: 5 - DNA, 21 bp, synthetic construct
  source 1..21: mol_type = other DNA
cgaagcaaagcctacagcaac

SEQ ID NO: 6 - DNA, 25 bp, synthetic construct
  source 1..25: mol_type = other DNA
tgtcaaattgttcattgtttggcat

SEQ ID NO: 7 - DNA, 23 bp, synthetic construct
  source 1..23: mol_type = other DNA
actaggtgtgacagacacagcaa

SEQ ID NO: 8 - DNA, 22 bp, synthetic construct
  source 1..22: mol_type = other DNA
tgttgtcccgatcagcagtgtc

SEQ ID NO: 9 - DNA, 20 bp, synthetic construct
  source 1..20: mol_type = other DNA
aatgggccacaatcaatcct

SEQ ID NO: 10 - DNA, 23 bp, synthetic construct
  source 1..23: mol_type = other DNA
ataacatagagcctgccttctgc

SEQ ID NO: 11 - DNA, 24 bp, synthetic construct
  source 1..24: mol_type = other DNA
ttcatctgtatcctcagagatccc

SEQ ID NO: 12 - DNA, 22 bp, synthetic construct
  source 1..22: mol_type = other DNA
ctcatctgagcttcagcatcac

SEQ ID NO: 13 - DNA, 23 bp, synthetic construct
  source 1..23: mol_type = other DNA
ctgaagagagamgrctggcyaag

SEQ ID NO: 14 - DNA, 24 bp, synthetic construct
  source 1..24: mol_type = other DNA
agctggagcaaattccatcaattc

SEQ ID NO: 15 - DNA, 28 bp, synthetic construct
  source 1..28: mol_type = other DNA
catayaarcatgctatattaaaagagtc

SEQ ID NO: 16 - DNA, 26 bp, synthetic construct
  source 1..26: mol_type = other DNA
gtttcttagaatctgctgtactctct

SEQ ID NO: 17 - DNA, 19 bp, synthetic construct
  source 1..19: mol_type = other DNA
aggacgcytcggagtacct

SEQ ID NO: 18 - DNA, 22 bp, synthetic construct
  source 1..22: mol_type = other DNA
gctaggacctctatcaagcacc

SEQ ID NO: 19 - DNA, 25 bp, synthetic construct
  source 1..25: mol_type = other DNA
ttagcaaagtcaagytgaatgatac

SEQ ID NO: 20 - DNA, 25 bp, synthetic construct
  source 1..25: mol_type = other DNA
ctttaagtatctttatagtgtcttc

SEQ ID NO: 21 - DNA, 23 bp, synthetic construct
  source 1..23: mol_type = other DNA
gaagagacactggcagacaactc

SEQ ID NO: 22 - DNA, 25 bp, synthetic construct
  source 1..25: mol_type = other DNA
aacattagctaagtgtctacggtac

SEQ ID NO: 23 - DNA, 21 bp, synthetic construct
  source 1..21: mol_type = other DNA
gcctctcttgttcttgctcgc

SEQ ID NO: 24 - DNA, 21 bp, synthetic construct
  source 1..21: mol_type = other DNA
caggtaagcgtaaaactcatc

SEQ ID NO: 25 - DNA, 19 bp, synthetic construct
  source 1..19: mol_type = other DNA
ggcactgaggacccacgtt

SEQ ID NO: 26 - DNA, 22 bp, synthetic construct
  source 1..22: mol_type = other DNA
ttgcgacatacccataaaagca

SEQ ID NO: 27 - DNA, 26 bp, synthetic construct
  source 1..26: mol_type = other DNA
caacaatggggttttacaggtaacct

SEQ ID NO: 28 - DNA, 24 bp, synthetic construct
  source 1..24: mol_type = other DNA
gtcaacacgcttaacaaagcactc

SEQ ID NO: 29 - DNA, 19 bp, synthetic construct
  source 1..19: mol_type = other DNA
cctyctcagcaacacgctc

SEQ ID NO: 30 - DNA, 19 bp, synthetic construct
  source 1..19: mol_type = other DNA
gaggaggtggtcttgatgc

SEQ ID NO: 31 - DNA, 19 bp, synthetic construct
  source 1..19: mol_type = other DNA
ggtcaccaacatggttctg

SEQ ID NO: 32 - DNA, 19 bp, synthetic construct
  source 1..19: mol_type = other DNA
atacacaagcccagctccc

SEQ ID NO: 33 - DNA, 18 bp, synthetic construct
  source 1..18: mol_type = other DNA
cgagcttagtcgtccacc

SEQ ID NO: 34 - DNA, 21 bp, synthetic construct
  source 1..21: mol_type = other DNA
gatttgaccctgtctcattac

SEQ ID NO: 35 - DNA, 19 bp, synthetic construct
  source 1..19: mol_type = other DNA
ctacacctcgctgctrgac

SEQ ID NO: 36 - DNA, 20 bp, synthetic construct
  source 1..20: mol_type = other DNA
gacatctgaaacatrgccgc

SEQ ID NO: 37 - DNA, 19 bp, synthetic construct
  source 1..19: mol_type = other DNA
actgaggagggcatgaagc

SEQ ID NO: 38 - DNA, 19 bp, synthetic construct
  source 1..19: mol_type = other DNA
gaagaggaggtggtaagcg

SEQ ID NO: 39 - DNA, 21 bp, synthetic construct
  source 1..21: mol_type = other DNA
agtaagacgggatataatgcc

SEQ ID NO: 40 - DNA, 20 bp, synthetic construct
  source 1..20: mol_type = other DNA
tacacatctgtgacgctacc

SEQ ID NO: 41 - DNA, 21 bp, synthetic construct
  source 1..21: mol_type = other DNA
tcggaactcctaatacgattc

SEQ ID NO: 42 - DNA, 21 bp, synthetic construct
  source 1..21: mol_type = other DNA
tgccataagaaacaggtacag

SEQ ID NO: 43 - DNA, 24 bp, synthetic construct
  source 1..24: mol_type = other DNA
acgccctatcgcaacacatttcac

SEQ ID NO: 44 - DNA, 19 bp, synthetic construct
  source 1..19: mol_type = other DNA
gggaagtgttcctsctgag

SEQ ID NO: 45 - DNA, 24 bp, synthetic construct
  source 1..24: mol_type = other DNA
tacgctcaccgtgcccagtgaacg

SEQ ID NO: 46 - DNA, 24 bp, synthetic construct
  source 1..24: mol_type = other DNA
tcatgattcgaacaaaggtgtaac

SEQ ID NO: 47 - DNA, 25 bp, synthetic construct
  source 1..25: mol_type = other DNA
ggattatgcctcccttaggtcacta

SEQ ID NO: 48 - DNA, 24 bp, synthetic construct
  source 1..24: mol_type = other DNA
tcaggaggggatggtgcctaccat

SEQ ID NO: 49 - DNA, 24 bp, synthetic construct
  source 1..24: mol_type = other DNA
gaagcgggatctatcacctaggct

SEQ ID NO: 50 - DNA, 26 bp, synthetic construct
  source 1..26: mol_type = other DNA
gttcgcaccaggcaactatcctgcca

SEQ ID NO: 51 - DNA, 24 bp, synthetic construct
  source 1..24: mol_type = other DNA
agcagcagggcaggatgttagaga

SEQ ID NO: 52 - DNA, 25 bp, synthetic construct
  source 1..25: mol_type = other DNA
gatgtaggcaccacaactgcagtga

SEQ ID NO: 53 - DNA, 25 bp, synthetic construct
  source 1..25: mol_type = other DNA
ctggtgcagttcgcccgtgccaccg

SEQ ID NO: 54 - DNA, 25 bp, synthetic construct
  source 1..25: mol_type = other DNA
taaggatcagctgctgtcatccagc

SEQ ID NO: 55 - DNA, 25 bp, synthetic construct
  source 1..25: mol_type = other DNA
tccgacacagtggggagagaggctc

SEQ ID NO: 56 - DNA, 25 bp, synthetic construct
  source 1..25: mol_type = other DNA
cacttgctgcaacttatcacaccgt

SEQ ID NO: 57 - DNA, 24 bp, synthetic construct
  source 1..24: mol_type = other DNA
ctgagcttgctcctacagccagtg

SEQ ID NO: 58 - DNA, 24 bp, synthetic construct
  source 1..24: mol_type = other DNA
gtccatggtaatgcacatgtagct

SEQ ID NO: 59 - DNA, 23 bp, synthetic construct
  source 1..23: mol_type = other DNA
acacctccgagagcagagccgca

SEQ ID NO: 60 - DNA, 23 bp, synthetic construct
  source 1..23: mol_type = other DNA
actctccgctccacaacgaggac

SEQ ID NO: 61 - DNA, 24 bp, synthetic construct
  source 1..24: mol_type = other DNA
accgtcgcttggctcacttaacag

SEQ ID NO: 62 - DNA, 23 bp, synthetic construct
  source 1..23: mol_type = other DNA
tcccacccagactagcccccagg

SEQ ID NO: 63 - DNA, 26 bp, synthetic construct
  source 1..26: mol_type = other DNA
tccytgcccyaaccactacagcaaag

SEQ ID NO: 64 - DNA, 23 bp, synthetic construct
  source 1..23: mol_type = other DNA
tgcctccgtatctktacgaatgc

SEQ ID NO: 65 - DNA, 24 bp, synthetic construct
  source 1..24: mol_type = other DNA
acgccctatcgcaacacatttcac

SEQ ID NO: 66 - DNA, 23 bp, synthetic construct
  source 1..23: mol_type = other DNA
tcgctcctggatmacggggtctc

(19) United States
(12) Patent Application Publication
(10) Pub. No.: US 2025/0073723 A1
(43) Pub. Date: Mar. 6, 2025

(54) HOMOGENEOUS MULTIPLEX DETECTION DEVICE, OPERATION AND DETECTION METHODS

(71) Applicant: HANGZHOU ZHILINGLONG BIOTECHNOLOGY CO LTD, Hangzhou City (CN)

(72) Inventors: Xing SU, Hangzhou City (CN); Kai WU, Hangzhou City (CN)

(21) Appl. No.: 18/819,146

(22) Filed: Aug. 29, 2024

(30) Foreign Application Priority Data
Aug. 29, 2023 (CN) 202311099234.1

(51) Int. Cl. B01L 7/00; C12Q 1/6816

(52) U.S. Cl. CPC B01L 7/52; C12Q 1/6816; B01L 2200/0647; B01L 2200/16; B01L 2300/041; B01L 2300/0832; B01L 2300/1838

(57) ABSTRACT

The present invention relates to a homogeneous multiplex detection device, its operation and detection processes thereof. The device comprises, a reaction vessel, providing a reaction space with two ends opposite to each other, wherein the first end of the reaction vessel is an open end and the second end of the reaction vessel is a closed end, and being used for nucleic acid amplification reactions; a reaction chip, positioning inside the reaction space and on the first end of the reaction vessel, and having a reaction surface where a plurality of types of nucleic acid probes corresponding to a plurality of types of target molecules are immobilized, wherein a plurality of types of nucleic acid probes of the reaction chip are corresponding to the plurality of types of target molecules, and are used for detecting nucleic acid molecules produced by amplification of the target molecules inside the reaction vessel; and a lid, detachably arranged on the first end of the reaction vessel to close the reaction space. The present invention simplifies multiplex detection process, reduces the possibility of product contamination, increases detection efficiency, reduces costs of use, and increases the reliability of reactions and detections.

### RELATED APPLICATION

This application claims priority from CN Application No.: 202311099234.1 titled, “Homogeneous multiplex detection device, operation method thereof and detection method” filed on Aug. 29, 2023, which is herein incorporated by reference in its entirety.

### FIELD OF THE INVENTION

The present invention relates to biochemical reactions and, particularly, to a homogeneous multiplex detection device and methods of operation and detection thereof.

### SEQUENCE LISTING

The instant application contains a Sequence Listing submitted electronically in XML format which was created on Sep. 30, 2024, and modified on Oct. 1, 2024, using WIPO ST.26 sequence listing. The said XML filed is named as HZBL-001-01US_sequence.xml and has a size of 58.0 KB. The XML format is hereby incorporated by reference in its entirety.

### BACKGROUND OF THE INVENTION

Several amplification devices have been developed that can amplify target molecules. In addition, detection devices have been developed to detect target molecules. However, there is a need for multiplex amplification and multiplex detection of a plurality of types of target molecules to be performed simultaneously in the same liquid phase reaction system.

Currently, there are several technologies that use circular convection chambers for PCR amplification along with microarrays for detection. However, these devices involve complicated structures, is complex in processing the ring-shaped convection consumables, and difficult to seal, thus increasing the probability of contamination by amplification product; the detection throughput is low, which makes it difficult to achieve high-throughput sample detection; the production efficiency of the microarrays is low, which makes it difficult to achieve large scale mass production; and the efficiency of target molecule amplification is low; the use of exonuclease-free DNA polymerase leads to low fidelity in amplification, which makes it difficult to amplify a plurality of related target molecules efficiently.

Therefore, there is an urgent need for a homogeneous multiplex device that not only allows simultaneous multiplex amplification and multiplex detection of a plurality of types of target molecules in the same liquid-phase reaction system, but also reduces the likelihood of product contamination, increases the throughput of the detection, reduces the costs of use, and increases the reliability of reactions and detections.

### DESCRIPTION OF THE INVENTION

In order to solve the above problems in the prior art, the present invention provides a homogeneous multiplex detection device and its operation and detection methods, which not only can simultaneously perform multiplex amplification and multiplex detection of a plurality of target molecules in the same liquid phase reaction system and simplify the multiplex detection process, but also can reduce the possibility of product contamination, increase the efficiency of detection, reduce costs of use, and increase reliability of reaction and the detection. The target molecules in the present invention refers to nucleic acid molecules or nucleic acid fragments to be detected, which may be nucleic acid molecule fragments formed by amplifying the target nucleic acid molecules to be detected, or nucleic acid molecules to be detected without amplification.

The present invention provides a homogeneous multiplex detection device, said device comprising of:

a reaction vessel, which provides a reaction space, has a first end and a said second end opposite to the first, wherein said first end of said reaction vessel is an open end and said second end of said reaction vessel is a closed end, and is a vessel for nucleic acid amplification reactions.

a reaction chip, which is arranged inside said reaction space and located on the first end of said reaction vessel, wherein a plurality of types of nucleic acid probes corresponding to a plurality of types of target molecules, respectively, are immobilized on a surface of said reaction chip, wherein said nucleic acid probes are used for detecting nucleic acid molecules produced by amplification of said target molecules inside said reaction vessel;

a lid, which is detachably arranged on said first end of said reaction vessel to close said reaction space.

In one embodiment of the present invention, the device further comprises a first heater and a second heater, said first heater being arranged on said first end of said reaction vessel, and said second heater being arranged on said second end of said reaction vessel, to allow for the formation of thermal convection in said reaction vessel when used for nucleic acid amplification reactions.

In one embodiment of the present invention, the device further comprises a third heater that is arranged between said first end and said second end of said reaction vessel.

In one embodiment of the present invention, when the reaction vessel is heated by both said first heater and said second heater, or by both said first heater and said second heater plus said third heater, the reaction vessel has a temperature between 30° C. and 75° C. on said first end of said reaction vessel and a temperature between 35° C. and 110° C. on said second end of said reaction vessel.

In one embodiment of the present invention, the reaction vessel is a tubular structure, and the first end of said reaction vessel and the second end of said reaction vessel are opposite to each other in a lengthwise direction of said tubular structure, wherein the first end and said second end of said reaction vessel can be concentrical or non-concentrical, and have the same or different cross-sections.

In an embodiment of the present invention, the cross-sections of the first end and the second end of said reaction vessel comprise at least one of a curved side and a straight side, respectively.

In an embodiment of the present invention, the cross sections of the first end and the second end of said reaction vessel have an inner diameter or minimum side length of from 0 0.5 mm to 5 mm, and said tubular structure has a length of from 5 mm to 50 mm, and said reaction space has a volume of from 5 μl to 5000 μl.

In one embodiment of the present invention, the reaction chip is provided in said tubular structure in a snap-fit or glued manner inside said tubular structure.

In one embodiment of the present invention, the reaction chip and said lid are integrated.

In one embodiment of the present invention, the reaction surface of said reaction chip is oriented in a radial direction towards said tubular structure direction, or towards lengthwise direction of said tubular structure and said second end of said reaction vessel.

In one embodiment of the present invention, the number of said plurality of types of nucleic acid probes is 2, 3 to 10, 3 to 20, 3 to 30, 3 to 300, 3 to 3000, or 3 to 3,000,000.

In one embodiment of the present invention, the plurality of types of nucleic acid probes are immobilized onto the reaction surface of said reaction chip by in situ synthesized or ex situ synthesis.

In one embodiment of the present invention, the first heater is integrated into said reaction chip.

In one embodiment of the present invention, the device further comprises a signal detector that detects a fluorescent signal, an optical signal or an electrical signal from a reaction chip.

In one embodiment of the present invention, the plurality of types of target molecules include one or more of RNA molecules or DNA molecules, RNA fragments in an RNA genome or DNA fragments in a DNA genome, and variant structures in RNA molecules or DNA molecules.

In one embodiment of the present invention, the plurality of types of target molecules include one or more of RNA virus nucleic acid molecules and DNA virus nucleic acid molecules, wherein said RNA virus includes one or more of influenza A virus InfA, influenza A virus H1N1 2009, influenza A virus H3N2, human parainfluenza virus HPIV1, human parainfluenza virus HPIV2, human parainfluenza virus HPIV3, human parainfluenza virus HPIV4, human metapneumovirus hMPV, respiratory adenovirus AdV, respiratory syncytial virus RSV, bocavirus BoV, severe acute respiratory syndrome coronavirus SARS-CoV, Middle East Respiratory Syndrome Coronavirus MERS-CoV, and one or more of Severe Acute Respiratory Syndrome Coronavirus 2SARS-CoV-2, wherein said DNA virus includes human herpesvirus HSV-1, human herpesvirus HSV-2, human herpesvirus VZV, human herpesvirus CMV, human herpesvirus EBV, human herpesvirus HHV-6, human herpesvirus HHV-7, and human herpesvirus HHV-8.

In one embodiment of the present invention, the plurality of types of target molecules are derived from humans, animals, plants, microorganisms or are synthesized artificially or chemically, wherein said microorganisms include one or more of viruses, bacteria, and fungi.

The present invention further provides a method for operating said homogeneous multiplex detection device, said method comprising:

adding a reaction system with a test sample and a reagent into the interior of a reaction space provided by a reaction vessel, said test sample include one or more target molecules to be detected.

heating the reaction vessel by the first heater and the second heater, so that the reaction system with a reagent and a test sample can form convection flow between the first end and the second end of the reaction vessel under the action of thermal convection, and the one or more target molecules to be detected in the test sample can hybridize not only with the complementary primers in the reaction system to achieve amplification, but also with the complementary nucleic acid probe molecules immobilized on the reaction surface of the reaction chip.

In one embodiment of the present invention, the method further comprises heating the reaction vessel with one or more third heaters.

In one embodiment of the present invention, both said first heater and said second heater, or both said first heater and said second heater plus said third heater are separately controlled so that a temperature on said first end of said reaction vessel is from 30° C. to 75° C. and a temperature on said second end of said reaction vessel is from 35° C. to 110° C.

In one embodiment of the present invention, said reaction vessel is vertically arranged or tilted.

In one embodiment of the present invention, when said reaction vessel is tilted, the angle between said reaction vessel and the vertical direction is at an angle of 0 to 45.

In one embodiment of the present invention, a reagent includes primers and at least one DNA polymerase.

In one embodiment of the present invention, the DNA polymerase has a 3′-5′ exonuclease activity.

The present invention further provides an detection method for use in said homogeneous multiplex detection device, comprising:

emitting excitation light by the light source and irradiating the reaction surface of the reaction chip, so that the nucleic acid molecules formed by amplifying one or more target molecules in the reaction vessel hybridize with the complementary nucleic acid probe molecules immobilized on the reaction surface of the reaction chip to generate fluorescent signals.

detecting said fluorescent signals by a fluorescent signal detector;

identifying the type of one or more target molecules that hybridize with the complementary nucleic acid probe molecules, based on the positions or types of the corresponding nucleic acid probes where the fluorescent signals are detected.

In one embodiment of the present invention, a fluorescent signal is generated by one or more of the methods of direct fluorescent dye excitation, dye intercalation, fluorescence resonance energy transfer, and fluorescence dequenching.

In one embodiment of the present invention, the types of the nucleic acid probes are identified by the locations of nucleic acid probes that are pre-defined on the reaction surface of a reaction chip.

The present invention further provides a detection method for use in said homogeneous multiplex detection device, comprising:

forming closed circuits, respectively, between a plurality of nucleic acid probes immobilized on a reaction surface of a reaction chip and a power source, so that the nucleic acid molecules formed by amplifying one or more target molecules in the reaction vessel can generate light signals after hybridization of the amplified target molecules with the corresponding nucleic acid probe molecules.

detecting said light signals by a light signal detector;

identifying the type of one or more target molecules that hybridize with the corresponding nucleic acid probe molecules, based on the positions or types of the corresponding nucleic acid probes where optical signals are detected.

In one embodiment of the present invention, the type of a nucleic acid probe is identified by the positions of the pre-defined nucleic acid probes on the reaction surface of a reaction chip.

The present invention further provides a detection method for use in said homogeneous multiplex detection device, said method comprising:

forming closed circuits, respectively, between a plurality of nucleic acid probes immobilized on a reaction surface of a reaction chip and an electrical signal detector;

emitting excitation light by a light source and irradiating said reaction surface of said reaction chip, so that the nucleic acid molecules formed by amplifying one or more target molecules in the reaction vessel can generate electrical signals after hybridization of the amplified target molecules with the corresponding nucleic acid probe molecules;

detecting electrical signals by said electrical signal detector;

identifying the type of one or more target molecules that hybridize with the corresponding nucleic acid probe molecules, based on the positions or types of the corresponding nucleic acid probes where electrical signals are detected.

In one embodiment of the present invention, the electrical signal is generated by a photoelectric effect, wherein a photosensitizer realizing the photoelectric effect is introduced into a corresponding nucleic acid probe by an amplified target molecule.

In one embodiment of the present invention, the type of the nucleic acid probe is identified by the pre-defined locations of the nucleic acid probes on the reaction surface of a reaction chip.

As described above, the present invention has the following beneficial effects:

In present invention, a reaction chip that can be used for multiplex detection is arranged inside a reaction vessel that can perform amplification reaction under the action of thermal convection, such that hybridization of probe molecules and target molecules occurs while performing multiplex amplification, and observing optical signals or electrical signals from a reaction chip. In the entire detection process, there is only one liquid phase, one reaction system, and one-step operation. In addition, the present invention allows reading chip information by means of electrical signals, effectively shielding background noises and providing a higher signal-to-noise ratio, and thus increasing the reliability of the detection results.

### BRIEF DESCRIPTION OF THE DRAWINGS

FIG. 1A is a schematic diagram of the overall structure of a homogeneous multiplex detection device of an embodiment of the present invention.

FIG. 1B is a side view of a homogeneous multiplex detection device of one embodiment of the present invention.

FIG. 2A is a schematic diagram of the overall structure of a homogeneous multiplex detection device of another embodiment of the present invention.

FIG. 2B is a side view of a homogeneous multiple detection device of another embodiment of the present invention.

FIGS. 3A to 3G are schematic diagrams, respectively, of a reaction chip in a homogeneous multiplex detection device of an embodiment of the present invention.

FIGS. 4A to 4D are schematic and sketch diagrams, respectively, of a detection method for a homogeneous multiplex detection device of an embodiment of the present invention.

### SPECIFIC EMBODIMENTS

Exemplary embodiments of the present invention are described below in accordance with the accompanying drawings.

Exemplary embodiments will be described herein in detail, examples of which are represented in the accompanying drawings. The following description relates to the accompanying drawings, the same numerals in different drawings indicate the same or similar elements unless otherwise indicated. The embodiments described in the following exemplary embodiments do not represent all embodiments consistent with the present invention. Rather, they are examples of devices and methods that are consistent with some aspects of the present invention as detailed in the appended claims.

The terms used in this invention are used solely for the purpose of describing specific embodiments only and are not intended to limit this invention. The singular forms of “a,” “said,” and “the” used in this invention and the appended claims are also intended to encompass the plural forms, unless the context clearly indicates otherwise. It should also be understood that the terms “and/or” as used herein refer to and encompass any or all possible combinations of one or more of the associated listed items. “Comprise” or “comprising” and similar words mean that the elements or objects appearing before “comprise” or “comprising” include the elements or objects listed after “comprise” or “comprising” and their equivalents, and do not exclude other elements or objects.

### First Embodiment of the Invention

A first embodiment of the present invention provides a homogeneous multiplex detection device.

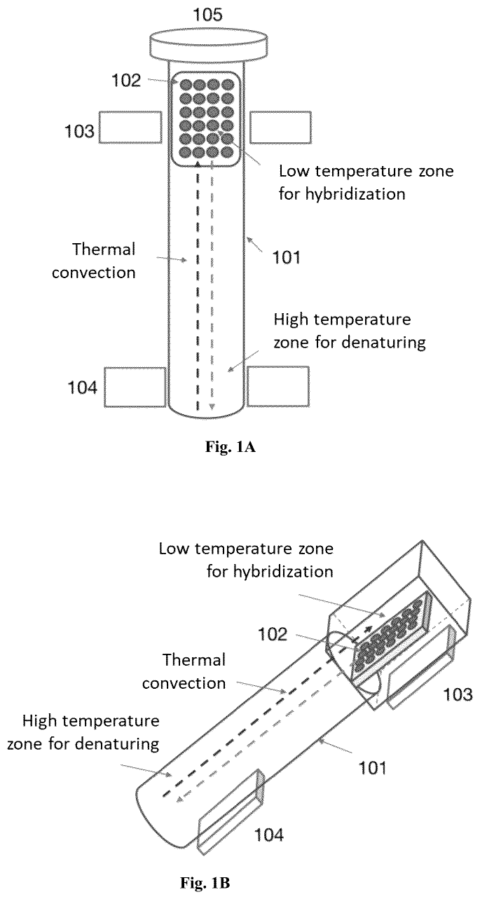

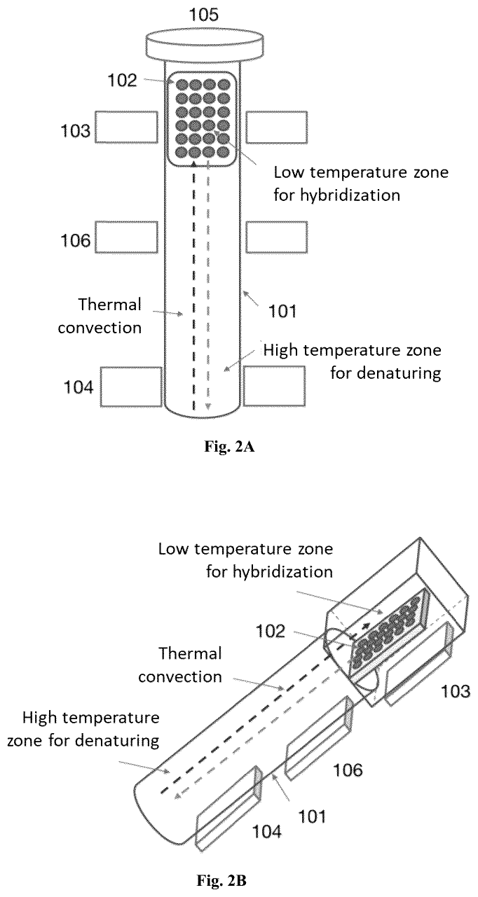

FIG. 1A is a schematic diagram of the overall structure of a homogeneous multiplex detection device of an embodiment of the present invention, and FIG. 1B is a side view of a homogeneous multiplex detection device of an embodiment of the present invention.

As shown in FIGS. 1A and 1B, the homogeneous multiplex detection device may comprise a reaction vessel 101, a reaction chip 102, and a lid 105

The reaction vessel 101 may also be referred to as a reaction test tube, the reaction vessel 101 may provide a reaction space, which may also be referred to as a reaction chamber, and the reaction vessel 101 may have a first end as well as a second end opposite to the first end, wherein the first end of the reaction vessel 101 may be an open end and the second end of the reaction vessel 101 may be a closed end. The reaction vessel 101 may be a vessel for a nucleic acid amplification reaction.

The lid 105 may be detachably arranged on the first end of the reaction vessel 101 to close off the reaction space, and the lid 105 may not be in contact with the reaction system in the reaction space, thereby greatly reducing the possibility of contamination due to product leakage.

In one embodiment, the reaction vessel 101 may be a tubular structure, such as a cylindrical structure or a rectangular cubic structure, which is more easily encapsulated than a reaction vessel with a sheet-like structure. The first end and the second end of the reaction vessel 101 may be arranged opposite to each other in a lengthwise direction of the tubular structure, and in FIGS. 1A and 1B, the first end of the reaction vessel 101 may also be referred to as the upper end of the tubular structure, and the second end of the reaction vessel 101 may also be referred to as the lower end of the tubular structure.

In one embodiment, the first end and the second end of the reaction vessel 101 may be aligned concentrically or non-concentrically.

In an embodiment, the first end and the second end of the reaction vessel 101 may have the same or different shapes of cross-sections.

In an embodiment, the cross sections of the first end and the second end of the reaction vessel 101 may comprise at least one of a curved side and a straight side, respectively.

In one embodiment, the cross-sections of the first end and the second end of the reaction vessel 101 may include a circular shape or/and a squared shape, respectively. In other words, the cross sections of the first end and the second end of the reaction vessel 101 may both be circular, such that the reaction vessel 101 may be a cylindrical structure. Alternatively, the cross-sections of the first end and the second end of the reaction vessel 101 may both be squared, such that the reaction vessel 101 may be a squared tubular structure. Alternatively, one of the first end and the second end of the reaction vessel 101 may have a circular cross-section and the other may have a squared cross-section, such that the reaction vessel 101 may have a profiled tubular structure.

In one embodiment, the inner diameter or the minimum side length of the cross section of the first end and the second end of the reaction container 101 may be 0.5 mm to 5 mm, the length of the tubular structure may be 5 mm to 50 mm, and the volume of the reaction space may be 5 μl to 5000 μl.

In one embodiment, the reaction vessel 101 may be made of a heat-resistant material, which may have a heat-resistant temperature of for example, greater than 150° C.

In one embodiment, the reaction vessel 101 may be made of a transparent material or an opaque material. As will be described in detail below, the transparent material may be suitable for fluorescent signal detection, optical signal detection, and the opaque material may be suitable for electrical signal detection.

Compared to a ring-shaped convection chamber, the tubular reaction vessel 101 is easier to process, such as being formed in one step using a conventional injection molding process, having a low production cost and being more reliable in use.

The reaction chip 102 may be arranged inside the reaction space and located on the first end of the reaction vessel 101.

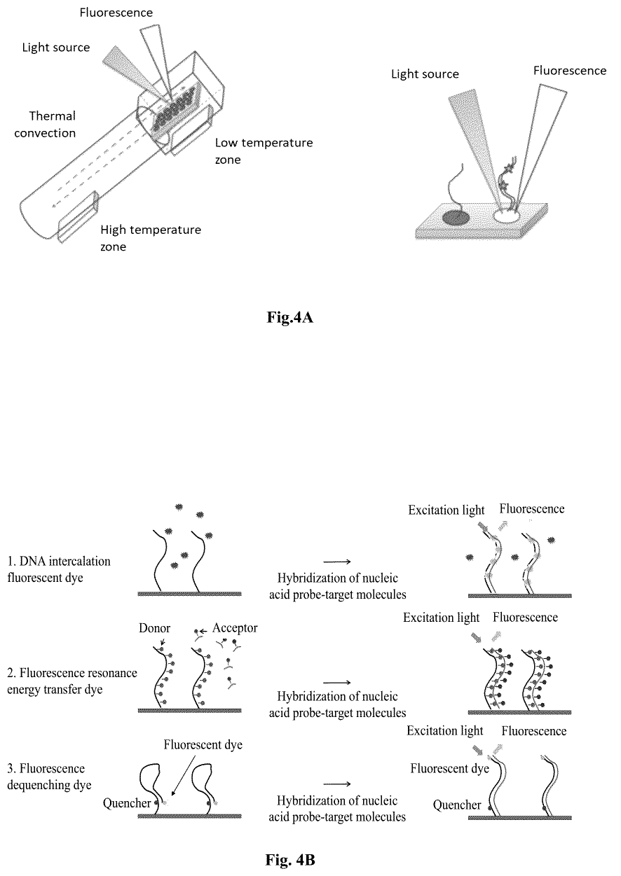

FIGS. 3A to 3G are schematic diagrams of the configuration of a reaction chip in a homogeneous multiplex detection device according to an embodiment of the present invention.

In one embodiment, the reaction chip 102 may be provided in the tubular structure in a snap-fit or glued manner.

As shown in FIG. 3A, the top view indicates that the reaction chip 102 can be inserted into the reaction vessel 101 as a tubular structure through the groove in the tubular structure.

As shown in FIG. 3B, the top view indicates that the reaction chip 102 can be bonded to the reaction vessel 101 as a tubular structure via an adhesive.

It will be appreciated by those skilled in the art that the reaction chip 102 may also be arranged in the tubular structure by force-fit or form-fit or other methods. As an example, the reaction chip 102 may be pre-connected or supported in the tubular structure by other connection structures or other support structures, and may be removed from the tubular structure as needed. As another example, the width of the reaction chip 102 may be equal to or slightly larger than the inner diameter of the tubular structure, such that the reaction chip 102 may be pre-snapped into the tubular structure and removed from the tubular structure as needed.

The dimensions of the reaction chip 102 and its position on the first end of the reaction vessel 101 can be rationally configured such that the reagent and test samples injected inside can cover the reaction surface of the reaction chip 102 and flow freely across the reaction surface of the reaction chip 102 under action of thermal convection.

In one embodiment, the reaction chip 102 can be made of silicon, glass, or high molecular weight polymer, which has low production cost and is suitable for large-scale production.

In one embodiment, a plurality of types of nucleic acid probes corresponding to a plurality of types of target molecules, respectively, may be immobilized on the reaction surface of reaction chip 102. The nucleic acid probes are used for detecting nucleic acid molecules produced by amplification of the target molecules inside the reaction vessel 101, and may form a two-dimensional probe array so that the x-y coordinate positions of different types of nucleic acid probes can be used as the labels for the different types of nucleic acid probes. As an example, the nucleic acid probe at position (1, 1) on the reaction surface may be pre-defined for type A, and the nucleic acid probe at position (1, 2) on the reaction surface may be pre-defined for type B, and so on.

The molecules of the nucleic acid probes may be single-stranded and form double-stranded structures when a probe molecule hybridizes (i.e., pairs) with a corresponding type of target molecules in the test sample, such that single-stranded molecules may be differentiated from the double-stranded molecules by fluorescent signals, optical signals, or electrical signals. In this way, when it is determined that a known nucleic acid probe molecule at a certain location on the reaction surface has hybridized with a corresponding type of a target molecule to form a double-stranded structure, the presence of the corresponding type of a target molecule in the test sample can be identified. If a plurality of nucleic acid probes corresponding to a plurality of types of target molecules are immobilized on the reaction surface, the presence of a plurality of types of target molecules in the test sample can be identified, thus realizing multiplex detection.

The nucleic acid probes can be immobilized onto the reaction surface of the reaction chip 102 either by in situ synthesis, i.e., directly synthesized and immobilized, or by ex situ synthesis, i.e., synthesized first then transferred for immobilization, and multiple known methods for synthesis and surface immobilization of nucleic acid probes exist and will not be restated herein.

In one embodiment, the number of the plurality of nucleic acid probes may be 2, 3 to 10, 3 to 20, 3 to 30, 3 to 300, 3 to 3000, or 3 to 3,000,000. As shown in FIGS. 1A and 1B, the number of the plurality of nucleic acid probes may be 24 and may be formed into a 4×6 probe array.

In one embodiment, the reaction surface of the reaction chip 102 may be oriented in a radial direction of the tubular structure, i.e., the reaction chip 102 may be arranged vertically in the tubular structure. In another embodiment, the reaction surface of the reaction chip 102 may be oriented in a lengthwise direction of the tubular structure and toward a second end of the reaction vessel, i.e., the reaction chip 102 may be horizontally arranged in the tubular structure.

In one embodiment, the reaction chip 102 may be pre-positioned in the tubular structure and may not be attached to the lid 105 as described above. In another embodiment, the reaction chip 102 may be integrated with the lid 105 and may enter the tubular structure with the lid 105 when the reaction space is closed by the lid 105. As an example, the reaction chip 102 may be vertically secured to the lid 105 such that a reaction surface of the reaction chip 102 may be oriented in a radial direction of the tubular structure when the reaction space is closed by the lid 105. As another example, the reaction chip 102 may be secured horizontally to the lid 105 such that, when the reaction space is closed by the lid 105, the reaction surface of the reaction chip 102 may be oriented in a lengthwise direction of the tubular structure and toward the second end of the reaction vessel 101.

As shown in FIG. 3C, the reaction chip 102 may be pre-arranged in the tubular structure and may be separated from the lid 105. The reaction surface of the reaction chip 102 may be oriented in a radial direction of the tubular structure, i.e., the reaction chip 102 may be vertically oriented in the tubular structure.

As shown in FIG. 3D, the reaction chip 102 may be vertically secured to the lid 105 so that it may enter the tubular structure with the lid 105 when the reaction space is closed by the lid 105. The reaction surface of the reaction chip 102 may thus be oriented in a radial direction of the tubular structure.

As shown in FIG. 3E, the reaction chip 102 can be secured horizontally anchored to the lid 105 such that it can enter the tubular structure with the lid 105 when the reaction space is closed by the lid 105. The reaction surface of the reaction chip 102 may be oriented in a lengthwise direction of the tubular structure and toward the second end of the reaction vessel 101.

Returning to FIGS. 1A and 1B, the homogeneous multiplex detection device may further comprise a first heater 103 and a second heater 104. The first heater 103 may be arranged on the first end of the reaction vessel 101, and the second heater 104 may be arranged on the second end of the reaction vessel 101 to allow the formation of thermal convection in the reaction vessel 101 when used for nucleic acid amplification reactions. Compared to a ring-shaped convection chamber, the tubular structure of the reaction vessel 101 has smaller contact areas with the first heater 103 and the second heater 104, and the heaters that are compatible with the tubular structure take up less space, allowing more reaction vessels 101 to be arranged for the same given volume, thereby enabling high-throughput sample detection.

In one embodiment, the first heater 103 and the second heater 104 are both capable of heating the reaction vessel 101 so that the reagent and the test sample added inside the reaction vessel can form a convective flow between the first end and the second end of the reaction vessel 101 under the action of thermal convection, so that one or more target molecules to be detected in the test sample can hybridize not only with the complementary primers of the reaction system to achieve amplification, but also with the complementary nucleic acid probe molecules immobilized on the reaction surface of the reaction chip 102 to achieve detection.

In one embodiment, the heating of both the first heater 103 and the second heater 104 may be controlled separately such that the temperature on the first end of the reaction vessel 101 may be lower than the temperature on the second end of the reaction vessel 101. Understandably, the higher the heating temperature, the lower the density (lower specific gravity) of the heated reaction system containing the reagent and the test sample, while the lower the heating temperature the higher the density (higher specific gravity) of the heated reaction system containing the reagent and the test sample. In this way, the reaction system at the first end of the reaction vessel 101 and the reaction system on the second end of the reaction vessel 101 can form thermal convection that results in constant flow and mixing of the liquids, promoting the hybridization process to be described below.

In one embodiment, the heating of the first heater 103 can be controlled so that free target molecules in the reaction system can hybridize with free primers and complementary nucleic acid probe molecules immobilized on the reaction surface of the reaction chip 102, and the heating of the second heater 104 can be controlled so that double-stranded target molecules in the reaction system be denatured into single-stranded molecules, and because of the low specific gravity of the second end, the single-stranded molecules flow to the first end, thereby, achieving exponential amplification.

In one embodiment, the heating of the first heater 103 can be controlled such that the temperature on the first end of the reaction vessel 101 can be between 30° C. and 75° C., and the heating of the second heater 104 can be controlled such that the temperature on the second end of the reaction vessel 101 can be between 30° C. and 110° C. (no boiling, i.e., no bubbles at 110° C. at 1 0.5 atmospheres). Notably, in known methods, the temperature in the high heat zone is less than 100° C., such as 95° C. Because the reaction vessel employed in the present invention is able to withstand high pressures, i.e., the liquid does not boil at greater than 100° C., and since the enzyme employed in the present invention is able to withstand high temperatures, i.e., it is not denatured at greater than 100° C., the temperature in the high heat zone of the present invention may be greater than or equal to 100° C., such as 110° C.

In yet another embodiment, the heating of the first heater 103 may be controlled so that the temperature on the first end of the reaction vessel 101 may be between 30° C. and 75° C., and the heating of the second heater 104 may be controlled so that the temperature on the second end of the reaction vessel 101 may be between 35° C. and 110° C.

In yet another embodiment, the heating of the first heater 103 may be controlled such that the temperature on the first end of the reaction vessel 101 may be at 65° C., and the heating of the second heater 104 may be controlled such that the temperature on the second end of the reaction vessel 101 may be at 98° C.

In one embodiment, the first heater 103 and the second heater 104 may both be arranged outside the reaction space. In another embodiment, the first heater 103 and the second heater 104 may both be arranged inside the reaction space. In yet another embodiment, one of the first heater 103 and the second heater 104 may be arranged outside the reaction space, while the other of the first heater 103 or the second heater 104 may be arranged inside the reaction space. As shown in FIGS. 1A and 1B, both the first heater 103 and the second heater 104 may be arranged outside the reaction space.

However, the first heater 103 may be integrated into the reaction chip 102 so that it may be arranged inside the reaction space, while the second heater 104 remains outside the reaction space.

As shown in FIG. 3F, the first heater 103 may include an insulation layer 202 and a substrate layer 204 and a heating layer 203 therebetween, and the reaction chip 102 with the nucleic acid probes 201 immobilized on the reaction surface is detachedly arranged above the insulating layer 202.

As shown in FIG. 3G, the first heater 103 may include the insulating layer 202 and the substrate layer 204 and the heating layer 203 therebetween, and the reaction chip 102 with the nucleic acid probes 201 immobilized on the reaction surface are integrated together with the insulating layer 202.

In one embodiment, the first heater 103 and the second heater 104 may be mono heaters to be arranged on the same side or different sides of the reaction vessel 101, respectively. In another embodiment, the first heater 103 and the second heater 104 may be dual heaters to be arranged on different sides of the reaction vessel 101. In yet another embodiment, the first heater 103 and the second heater 104 may be ring-shaped heaters to be arranged around the reaction vessel 101, respectively.

In one embodiment, each of the first heater 103 and the second heater 104 may be coupled to the reaction vessel 101 through one or more of contact heat transfer, radiation, thermal convection, electromagnetic induction, and the like.

In one embodiment, the first heater 103 and the second heater 104 may be of a resistance heater, or a PET heater, or a PI heater, or a silicone heater, among others. As an example, the first heater 103 may be of a resistance heater and may be integrated into the reaction chip 102 so that it may be arranged inside the reaction space, while the second heater 104 may be of a PET heater, or a PI heater, or a silicone heater and may be arranged outside the reaction space.

In one embodiment, the first heater 103 and the second heater 104 may be provided to each corresponding reaction vessel 101. In another embodiment, the first heater 103 and the second heater 104 may be provided to a plurality of reaction vessels 101 so that several to dozens of reactions may be controlled simultaneously, allowing for high throughput sample detection.

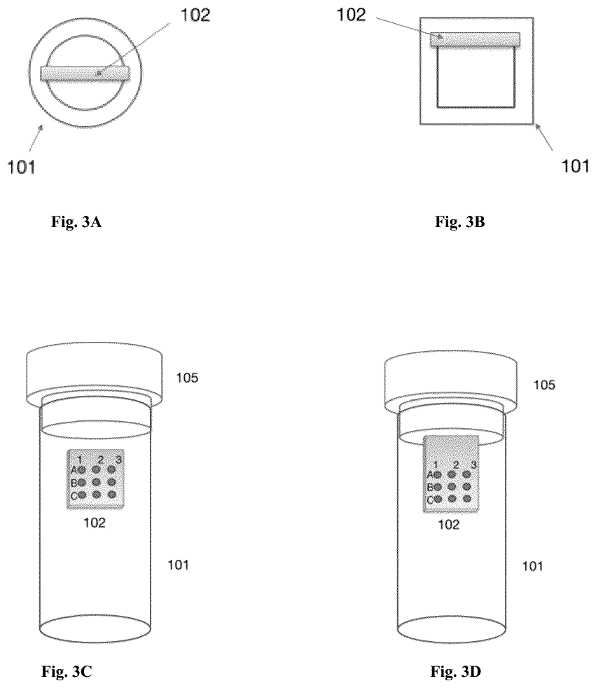

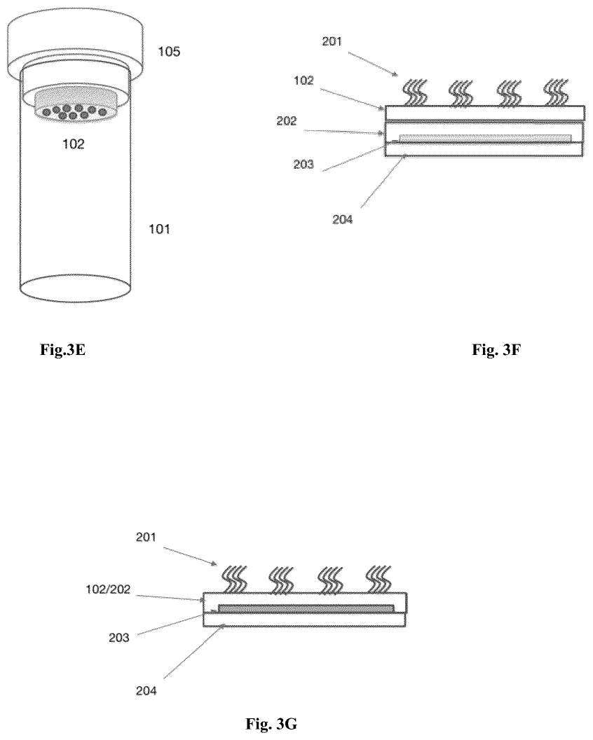

FIG. 2A is a schematic view of the overall structure of a homogeneous multiplex detection device of another embodiment of the present invention, and FIG. 2B is a side view of a homogeneous multiplex detection device of another embodiment of the present invention.

As shown in FIG. 2A and FIG. 2B, compared to FIG. 1A and FIG. 1B, the homogeneous multiplex detection device may further comprise a third heater 106, the third heater 106 may be one unit or multiple units, and the third heater 106 may be arranged between a first end and a second end of the reaction vessel 101. Compared to ring-shaped convection chamber, the tubular structure of the reaction vessel 101 has a smaller contact area with the first heater 103, the second heater 104, and the third heater 106, and the heaters that are compatible with the tubular structure occupy less space, allowing more reaction vessels 101 to be arranged in the same given space, thereby allowing for high throughput sample detection.

In one embodiment, the first heater 103 and the second heater 104 plus the third heater 106 are capable of heating the reaction vessel 101 such that the reagent and the test sample to be added inside the reaction vessel are able to form a convection flow between the first end and the second end of the reaction vessel 101 under the action of thermal convection, and such that one or more target molecules to be detected in the test sample are able to hybridize not only with complementary primers in the reaction system to achieve amplification, but also with complementary nucleic acid probe molecules immobilized on the reaction surface of the reaction chip 102 to achieve detection.

In one embodiment, the heating of each heater (the first heater 103, the second heater 104, and the third heater 106) may be controlled independently such that the temperature on the first end of the reaction vessel 101 may be lower than the temperature on the second end of the reaction vessel 101. It is understood that the higher the heating temperature, the lower the density (lower specific gravity) of the heated reaction system containing the reagent and the test sample, while the lower the heating temperature, the higher the density (higher specific gravity) of the heated reaction system containing the reagent and the test sample. In this way, the reagent and test sample located on the first end of the reaction vessel 101 and the reagents and test sample located on the second end of the reaction vessel 101 can form a thermal convection, resulting in constant flow and mixing of the liquids, promoting the hybridization process to be described below.

In one embodiment, the heating of the first heater 103, the second heater 104, and the third heater 106 can be controlled independently so that the free target molecules in the reaction system hybridize with the free primers and the corresponding nucleic acid probe molecules immobilized on the reaction surface of the reaction chip 102, and the double-stranded target molecules in the reaction system can be denatured to single-stranded molecules and flow to the first end due to the low specific gravity of the second end, thereby achieving exponential amplification.

In one embodiment, the heating of the first heater 103, the second heater 104 and the third heater 106 may be independently controlled such that the temperature on the first end of the reaction vessel 101 may be between 30° C. and 75° C., and such that the temperature on the second end of the reaction vessel 101 may be between 30° C. and 110° C. (no boiling, i.e. no bubbles, at 110° C. under 1 0.5 atmospheres). It is worth noting that in the known methods, the temperature of the high heat zone is often less than 100° C., such as 95° C. Because the reaction vessel used in the present invention can withstand high pressures, the liquid not boiling at greater than 100° C. under higher pressure, and the enzyme used in the present invention is also able to withstand high temperatures, not being denatured at greater than 100° C., the temperature of the high heat zone of the present invention can be greater than or equal to 100° C., such as 110° C.

In yet another embodiment, the heating of the first heater 103, the second heater 104, and the third heater 106 may be separately controlled such that the temperature on the first end of the reaction vessel 101 may be between 30° C. and 75° C., and such that the temperature on the second end of the reaction vessel 101 may be between 35° C. and 110° C.

In yet another embodiment, the heating of the first heater 103, the second heater 104, and the third heater 106 may be separately controlled such that the temperature on the first end of the reaction vessel 101 may be at 65° C., and such that the temperature on the second end of the reaction vessel 101 may be at 98° C.

In one embodiment, the first heater 103, the second heater 104, and the third heater 106 may all be arranged outside the reaction space. In another embodiment, the first heater 103, the second heater 104, and the third heater 106 may all be arranged inside the reaction space. In yet another embodiment, portions of the first heater 103, the second heater 104, and the third heater 106 may be arranged outside the reaction space, and other portions of the first heater 103, the second heater 104, and the third heater 106 may be arranged inside the reaction space. As shown in FIGS. 2A and 2B, the first heater 103, the second heater 104, and the third heater 106 may all be arranged outside the reaction space.

However, the first heater 103 may be integrated into the reaction chip 102 so that it may be arranged inside the reaction space, while the second heater 104 and the third heater 106 remain arranged outside the reaction space.

In one embodiment, the first heater 103, the second heater 104, and the third heater 106 may be mono heaters to be arranged on the same or different sides of the reaction vessel 101, respectively. In another embodiment, the first heater 103, the second heater 104, and the third heater 106 may be dual heaters to be arranged on different sides of the reaction vessel 101. In yet another embodiment, the first heater 103, the second heater 104, and the third heater 106 may be ring-shaped heaters to be placed around the reaction vessel 101, respectively.

In one embodiment, each of the first heater 103, the second heater 104, and the third heater 106 may be coupled to the reaction vessel 101 through one or more of contact heat conduction, radiation, thermal convection, electromagnetic induction, and the like.

In one embodiment, the first heater 103, the second heater 104, and the third heater 106 may be a resistance heater, or a PET heater, or a PI heater or a silicone heater, among others. As an example, the first heater 103 may be of a resistance heater and may be integrated into the reaction chip 102 so that it may be arranged inside the reaction space, while the second heater 104 and the third heater 106 may be a PET heater, or a PI heater or a silica gel heater and may be arranged outside the reaction space.

In one embodiment, the first heater 103, the second heater 104, and the third heater 106 may be provided to one single unit of reaction vessel 101. In another embodiment, the first heater 103, the second heater 104, and the third heater 106 may be provided to a multiple units of reaction vessels 101 such that several to dozens of reactions may be controlled simultaneously, allowing for high-throughput sample detection.

In one embodiment, the plurality of types of target molecules can include one or more of RNA molecules or DNA molecules, RNA fragments in an RNA genome or DNA fragments in a DNA genome, and variant structures in RNA molecules or DNA molecules.

In one embodiment, the variant structure can include Single Nucleotide Polymorphisms (SNPs).

In one embodiment, the plurality of types of target molecules can include one or more of RNA virus nucleic acid molecules and DNA virus nucleic acid molecules. In other words, the plurality of types of target molecules can include only RNA virus nucleic acid molecules, can include only DNA virus nucleic acid molecules, or can include both RNA virus nucleic acid molecules and DNA virus nucleic acid molecules.

RNA viruses may include one or more of the 14 common respiratory RNA viruses, including Influenza A virus InfA, Influenza A virus H1N1 2009, Influenza A virus H3N2, human parainfluenza virus HPIV1, human parainfluenza virus HPIV2, human parainfluenza virus HPIV3, human parainfluenza virus HPIV4, human metapneumovirus hMPV, respiratory adenovirus AdV, respiratory syncytial virus RSV, Boca virus BoV, severe acute respiratory syndrome coronavirus SARS-CoV, Middle East respiratory syndrome coronavirus MERS-CoV, and severe acute respiratory syndrome coronavirus 2 SARS-CoV2.

The DNA virus may include one or more of 8 common human herpes DNA viruses such as human herpesvirus HSV-1, human herpesvirus HSV-2, human herpesvirus VZV, human herpesvirus CMV, human herpesvirus EBV, human herpesvirus HHV-6, human herpesvirus HHV-7, and human herpesvirus HHV-8.

In one embodiment, the plurality of types of target molecules can be derived from a human, an animal, a plant, a microorganism, or organisms synthesized artificially or chemically, wherein the microorganism can include one or more of viruses, a bacterium, and a fungi.

The homogeneous multiplex detection device may further comprise a signal detector, which may detect optical signals or electrical signals from reaction chip 102.

### Second Embodiment

A second embodiment of the present invention provides a method for a homogeneous multiplex detection device described in the first embodiment above, said method comprising:

adding a reaction system containing a reagent and a test sample to the inside of a reaction space within a reaction vessel, wherein the test sample to be detected may include one or more target molecules;

heating the reaction vessel by the first heater and the second heater to enable the reaction system to flow between the first end and second end of the reaction vessel under the action of thermal convection, and to enable one or more target molecules to be detected in the test sample to hybridize not only with the complementary primers in the reaction system to achieve amplification, but also with complementary nucleic acid probe molecules immobilized on the reaction surface of the reaction chip.

In one embodiment, the heating of both the first heater and the second heater may be separately controlled such that the temperature on the first end of the reaction vessel may be lower than the temperature on the second end of the reaction vessel. It is understood that the higher the heating temperature, the lower the density (lower specific gravity) of the heated reaction system containing the reagent and the test sample, while the lower the heating temperature, the higher the density (higher specific gravity) of the heated reaction system containing the reagent and the test sample. In this way, the reaction system on the first end of the reaction vessel and the reaction system on the second end of the reaction vessel can form thermal convection that results in constant flow and mixing of the liquids, promoting the hybridization process to be described below.

In one embodiment, the heating of the first heater can be controlled so that free target molecules in reaction system hybridize with free primers and corresponding nucleic acid probe molecules immobilized on the reaction surface of the reaction chip, and the heating of the second heater can be controlled so that the double-stranded target molecules in the reaction system can be denatured into single-stranded molecules and flow to the first end due to the low specific gravity of the second end, thereby achieving exponential amplification.

In one embodiment, the heating of the first heater may be controlled such that the temperature on the first end of the reaction vessel may be between 30° C. and 75° C., and the heating of the second heater may be controlled such that the temperature on the second end of the reaction vessel may be between 30° C. and 110° C. (no boiling, i.e. no bubbles, at 110° C. under 1 0.5 atmospheres). Notably, in known methods, the temperature in the high heat zone is often less than 100° C., such as 95° C. Because the reaction vessel employed in the present invention is able to withstand high pressures, i.e. the liquid does not boil at greater than 100° C. (under a higher pressure), and the enzyme employed in the present invention is able to withstand high temperatures, i.e. it is not denatured at greater than 100° C., the temperature in the high heat zone of the present invention may be greater than or equal to 100° C., such as 110° C.

In yet another embodiment, the heating of the first heater may be controlled such that the temperature on the first end of the reaction vessel may be between 30° C. and 75° C., and the heating of the second heater may be controlled such that the temperature on the second end of the reaction vessel may be between 35° C. and 110° C.

In yet another embodiment, the heating of the first heater may be controlled such that the temperature on the first end of the reaction vessel may be at 65° C., and the heating of the second heater may be controlled such that the temperature on the second end of the reaction vessel may be at 98° C.

In one embodiment, the method of operation may further comprise that the reaction vessel may be heated by one or more units of the third heaters.

In one embodiment, the heating of the first heater, the second heater, and the third heater may be separately controlled such that the temperature on the first end of the reaction vessel may be lower than the temperature on the second end of the reaction vessel. It is understood that the higher the heating temperature, the lower the density (lower specific gravity) of the heated reaction system, while the lower the heating temperature, the higher the density (higher specific gravity) of the heated reaction system. In this way, the reaction system on the first end of the reaction vessel and reaction system on the second end of the reaction vessel can form thermal convection that results in constant flow and mixing of the liquids, promoting the hybridization process to be described below.

In one embodiment, the heating of the first heater, the second heater, and the third heater may be separately controlled so that free target molecules of the reaction system can hybridize with free primers as well as the corresponding nucleic acid probe molecules immobilized on the reaction surface of the reaction chip, and such that the double-stranded target molecules in the reaction system can be denatured into single-stranded molecules and flow to the first end because of the lower specific gravity of the liquid on the second end, thereby achieving exponential amplification.

In one embodiment, the heating of the first heater, the second heater and the third heater may be separately controlled such that the temperature on the first end of the reaction vessel may be between 30° C. and 75° C., and such that the temperature on the second end of the reaction vessel may be between 30° C. and 110° C. (no boiling, i.e. no bubbles, at 110° C. under 1.5 atmospheres).

Notably, in known methods, the temperature in the high heat zone is often less than 100° C., such as 95° C. Because the reaction vessel used in the present invention can withstand high pressures, i.e., the liquid would not boil at greater than 100° C. under higher pressure, and the enzyme used in the present invention is also able to withstand high temperatures, i.e., not being denatured at greater than 100° C., the temperature of the high heat zone of the present invention can be greater than or equal to 100° C., such as 110° C.

In another embodiment, the heating of the first heater, the second heater, and the third heater may be individually controlled such that the temperature on the first end of the reaction vessel may be 30° C. to 75° C., and such that the temperature on the second end of the reaction vessel may be 35° C. to 110° C.

In yet another embodiment, the heating of the first heater, the second heater, and the third heater may be separately controlled such that the temperature on the first end of the reaction vessel may be 65° C., and such that the temperature on the second end of the reaction vessel may be 98° C.

In one embodiment, the reaction vessel may be vertically arranged, and in another embodiment, the reaction vessel may be titled. As shown in FIGS. 1A and 2A, the reaction vessel 101 may be vertically oriented, and as shown in FIGS. 1B and 2B, the reaction vessel 101 may be titled. In yet another embodiment, the angle between the reaction vessel and the vertical direction is 0 to 45 degrees.

As used herein, a reagent refers to the liquid that is used to mix with a test sample and added into the interior of the reaction space provided by the reaction vessel to form a reaction system. In other words, a reaction system includes all liquid components in the interior of the reaction space during a detection test using the homogeneous multiplex detection device. The reaction system may serve at least two purposes, one is to provide a liquid enzymatic reaction environment for multiplex amplification, and the other is to provide conditions for molecular hybridization of the target molecules with the nucleic acid probes.

In one embodiment, a reaction system includes a plurality of pairs of primers corresponding to, respectively, a plurality of target molecules, and said primers are used to hybridize with the corresponding target molecules in the reaction system to achieve amplification, thereby increasing the concentrations of the target molecules by molecular amplification under the action of thermal convection, and increasing the probability for the target molecules to hybridize with the molecules of the nucleic acid probes immobilized on the reaction surface of the reaction chip. Molecular amplification may include polymerase chain reaction (PCR), loop-mediated isothermal amplification (LAMP), Nicking Enzyme Amplification Reaction (NEAR), Nucleic acid sequence-based amplification (NASBA), rolling-circle nucleic acid amplification (RCA), Helicase dependent amplification (HDA), recombinase polymerase amplification (RPA), and enzymatic recombinase amplification techniques (ERA).

As in Table 1 below, for different types of target molecules, complementary primers can be prepared to add into the reaction system, and complementary nucleic acid probes can be prepared for immobilization onto the reaction surface of the reaction chip.

Table 1 lists target molecules and their corresponding primer sequences and nucleic acid probe sequences

| Name of | | |
|---|---|---|
| target | | |
| molecules | Primer Sequences (5′→3′) | Probe Sequences (5′→3′) |
| InfA | F: AGACCAATYYTGTCACCTCTG | TACGCTCACCGTGCCC |
| | R: CGGGTCCCCATTCCCATT | AGTGAACG |
| H1N1 | F: GGTTTGAGATATTCCCCAARAC | TCATGATTCGAACAAA |
| 2009 | R: AGGATTTGCTGAGCTTTGGGTATG | GGTGTAAC |
| H3N2 | F: CGAAGCAAAGCCTACAGCAAC | GGATTATGCCTCCCTTA |
| | R: TGTCAAATTGTTCATTGTTTGGCAT | GGTCACTA |
| HPIV1 | F: ACTAGGTGTGACAGACACAGCAA | TCAGGAGGGGATGGTG |
| | R: TGTTGTCCCGATCAGCAGTGTC | CCTACCAT |
| HPIV2 | F: AATGGGCCACAATCAATCCT | GAAGCGGGATCTATCA |
| | R: ATAACATAGAGCCTGCCTTCTGC | CCTAGGCT |
| HPIV3 | F: TTCATCTGTATCCTCAGAGATCCC | GTTCGCACCAGGCAAC |
| | R: CTCATCTGAGCTTCAGCATCAC | TATCCTGCCA |
| HPIV4 | F: CTGAAGAGAGAMGRCTGGCYAAG | AGCAGCAGGGCAGGAT |
| | R: AGCTGGAGCAAATTCCATCAATTC | GTTAGAGA |
| Hmpv | F: CATAYAARCATGCTATATTAAAAGAGTC | GATGTAGGCACCACAA |
| | R: GTTTCTTAGAATCTGCTGTACTCTCT | CTGCAGTGA |
| AdV | F: AGGACGCYTCGGAGTACCT | CTGGTGCAGTTCGCCC |
| | R: GCTAGGACCTCTATCAAGCACC | GTGCCACCG |
| RSV | F: TTAGCAAAGTCAAGYTGAATGATAC | TAAGGATCAGCTGCTG |
| | R: CTTTAAGTATCTTTATAGTGTCTTC | TCATCCAGC |
| BoV | F: GAAGAGACACTGGCAGACAACTC | TCCGACACAGTGGGGA |
| | R: AACATTAGCTAAGTGTCTACGGTAC | GAGAGGCTC |
| SARS- | F: GCCTCTCTTGTTCTTGCTCGC | CACTTGCTGCAACTTAT |
| CoV | R: CAGGTAAGCGTAAAACTCATC | CACACCGT |
| MERS- | F: GGCACTGAGGACCCACGTT | CTGAGCTTGCTCCTACA |
| CoV | R: TTGCGACATACCCATAAAAGCA | GCCAGTG |
| SARS- | F: CAACAATGGGGTTTTACAGGTAACCT | GTCCATGGTAATGCAC |
| CoV-2 | R: GTCAACACGCTTAACAAAGCACTC | ATGTAGCT |
| HSV-1 | F: CCTYCTCAGCAACACGCTC | ACACCTCCGAGAGCAG |
| | R: GAGGAGGTGGTCTTGATGC | AGCCGCA |
| HSV-2 | F: GGTCACCAACATGGTTCTG | ACTCTCCGCTCCACAAC |
| | R: ATACACAAGCCCAGCTCCC | GAGGAC |
| VZV | F: CGAGCTTAGTCGTCCACC | ACCGTCGCTTGGCTCAC |
| | R: GATTTGACCCTGTCTCATTAC | TTAACAG |
| CMV | F: CTACACCTCGCTGCTRGAC | TCCCACCCAGACTAGC |
| | R: GACATCTGAAACATRGCCGC | CCCCAGG |
| EBV | F: ACTGAGGAGGGCATGAAGC | TCCYTGCCCYAACCAC |
| | R: GAAGAGGAGGTGGTAAGCG | TACAGCAAAG |
| HHV-6 | F: AGTAAGACGGGATATAATGCC | TGCCTCCGTATCTKTAC |
| | R: TACACATCTGTGACGCTACC | GAATGC |
| HHV-7 | F: TCGGAACTCCTAATACGATTC | ACGCCCTATCGCAACA |
| | R: TGCCATAAGAAACAGGTACAG | CATTTCAC |
| HHV-8 | F: ACGCCCTATCGCAACACATTTCAC | TCGCTCCTGGATMACG |
| | R: GGGAAGTGTTCCTSCTGAG | GGGTCTC |

In one embodiment, a reagent includes primers and DNA polymerase. In another embodiment, a reagent includes MgCl2 at a concentration of 3 mM, dNTP at a concentration of 0.2 mM, a plurality of primers, each at a concentration ranged from 0.1 μM to 0.6 μM, a DNA polymerase at a concentration of 0.05 U/μl, a RNA reverse transcriptase at a concentration of 0.5 U/μl, DTT at a concentration of 1 mM, Tween-20 at a concentration of 0.05%, Tris-HCl pH 8.8 at a concentration of 25 mM, and K2SO4 at a concentration of 30 mM. In yet another embodiment, the DNA polymerase has a 3′→5′ exonuclease activity. The use of a polymerase with 3′→5′ exonuclease activity can increase detection rate for mutated virus strains, repair mis-paired bases of primers, achieve higher fidelity, reduce non-specific amplification products, and thus resulting in more reliable results.

In one embodiment, the volume of the reaction system can be 50 μl.

It is understood that the above listed types of target molecules may be used for illustrative purposes only and are not intended to be limiting. In fact, of the actual needs, those skilled in the art can set up other types of target molecules and prepare corresponding primer sequences and nucleic acid probe sequences, and adjust the reaction system to achieve the effect of homogeneous multiplex amplification and multiplex detection of a plurality of target molecules in the same liquid-phase reaction system.

first embodiment is a device-related embodiment corresponding to the present embodiment, and the present embodiment can be implemented in conjunction with the first embodiment. The relevant technical details mentioned in the first embodiment are still valid in the present embodiment, and are not repeated herein to minimize repetition. Accordingly, the relevant technical details mentioned in the present embodiment can also be applied in the first embodiment.

### Third Embodiment

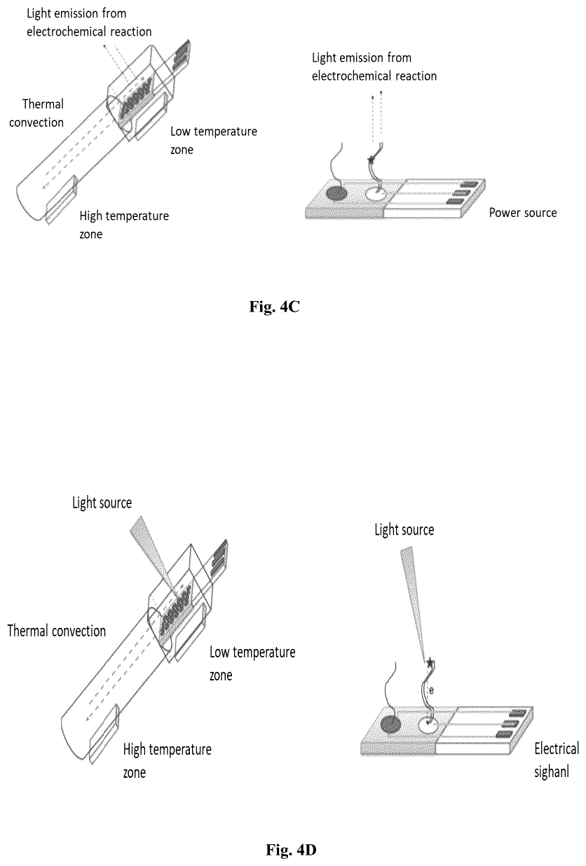

A third embodiment of the present invention provides a detection method for a homogeneous multiplex detection device. FIG. 4A is a schematic diagram of a detection method for a homogeneous multiplex detection device of an embodiment of the present invention. The homogeneous multiplex detection device has been described in the first embodiment above, and as shown in FIG. 4A, the detection method may comprise:

emitting an excitation light by a light source and irradiating onto a reaction surface of the reaction chip, such that nucleic acid molecules produced by amplification of one or more target molecules in the reaction vessel can generate fluorescent signals after hybridization of the amplified target molecules with corresponding nucleic acid probe molecules immobilized on the reaction surface of the reaction chip;

detecting the fluorescent signals by a fluorescent signal detector;

identifying the type of one or more target molecules that hybridize with the corresponding nucleic acid probe molecules based on the type of the corresponding nucleic acid probes where the fluorescent signals are detected.

In one embodiment, the light source may be a laser light source, or a LED light source or other light sources. When the excitation light emitted by the light source irradiates the reaction surface of the reaction chip, the double-stranded molecules formed by hybridization of the target molecules with the corresponding nucleic acid probe molecules can generate a fluorescent signal.

In one embodiment, a fluorescent signal can be generated by one or more of the methods of fluorescent dye direct excitation, dye intercalation, fluorescence resonance energy transfer, and fluorescence dequenching.

FIG. 4B is a schematic diagram of generating fluorescent signals by one or more of the methods of an embodiment of the present invention, including dye intercalation, fluorescence resonance energy transfer, and fluorescence dequenching.

As shown in FIG. 4B, for the dye-intercalation method, a double-stranded DNA molecule-specific intercalated fluorescent dye is used, e.g., including the EB, SYBR series, Gold View series, Gel Red, etc., that does not emit light or emits weak fluorescence when the dye is free, but emits a strong fluorescence (enhanced 20- to 30-fold) when the dye is intercalated into DNA double-helices, and thus is specifically detected.

As shown in FIG. 4B, for the fluorescence resonance energy transfer method, a fluorescent dye is used to stimulate fluorescence resonance energy transfer through hybridization. A dye can be classified as a donor dye or an acceptor dye. When a donor dye is attached to the nucleic acid probe molecule and an acceptor dye is attached to the target molecule, the donor dye and the acceptor dye are brought into proximity to each other only when a probe molecule and a target molecule are hybridized, resulting in the light emission of the acceptor dye when the donor dye is excited.

As shown in FIG. 4B, for the fluorescence dequenching method, a fluorescent dye introduced by hybridization is used. When a probe molecule is not hybridized with a target molecule, the fluorescent dye is affected by the adjacent quencher and does not emit light. When the probe molecule hybridizes with the target molecule, the quencher molecule is removed or displaced (for example, including TaqMan probe method, molecular beacon method, scorpion probe method, etc.), so that the fluorescent dye is de-quenched and emits light when excited.

In one embodiment, the fluorescent signal detector may be a CCD camera, or a EMCCD camera, or a CMOS camera or a photosensitive sensor (such as a photomultiplier tube), etc., and the fluorescent signals may be acquired and detected by means of light scanning or imaging, etc.

In one embodiment, the type of a nucleic acid probe can be identified by the pre-defined position of the nucleic acid probe on the reaction surface of the reaction chip. As already described in the first embodiment above, when it is determined that a molecule of a known nucleic acid probe at a certain position on the reaction surface hybridizes with a corresponding type of target molecule to form a double-stranded molecule, the presence of a corresponding type of target molecule in the test sample can be identified. When a plurality of nucleic acid probes complementary to each of the plurality of types of target molecules are immobilized on the reaction surface, it is possible to identify the presence of the plurality of types of target molecules in the test sample, thereby achieving a multiplex detection.

In one embodiment, the amount of the target molecules can be calculated from the kinetic data of the signals.

The first embodiment is a device embodiment corresponding to the present embodiment, and the present embodiment may be implemented in conjunction with the first embodiment. The relevant technical details mentioned in the first embodiment are still valid in the present embodiment, and are not repeated herein to minimize repetition. Accordingly, the technical details mentioned in the present embodiment can also be applied in the first embodiment.

### Fourth Embodiment

A fourth embodiment of the present invention provides a detection method for a homogeneous multiplex detection device. FIG. 4C is a schematic diagram of a detection method for a homogeneous multiplex detection device of an embodiment of the present invention. The homogeneous multiplex detection device has been described in the first embodiment above, and as shown in FIG. 4C the detection method may comprise:

forming closed circuits, respectively, by the plurality of nucleic acid probes immobilized on a reaction surface of a reaction chip and a power source, such that nucleic acid molecules formed by amplification of one or more target molecules in the reaction vessel can generate light signals after hybridization with corresponding nucleic acid probe molecules;

[detecting the light signals by a light signal detector;

identifying the type of one or more target molecules that hybridize with the corresponding nucleic acid probe molecules based on the type of the nucleic acid probes where the fluorescent signals are detected.

In one embodiment, the optical signal detector may be a CCD camera, or a EMCCD camera, or a CMOS camera or a photosensitive sensor (such as a photomultiplier tube), etc., and the optical signals may be captured and detected by means of optical scanning or imaging, etc.

In one embodiment, the type of a nucleic acid probe can be identified by the pre-defined position of the nucleic acid probe on the reaction surface of the reaction chip. As already described in the first embodiment above, when it is determined that a molecule of a known nucleic acid probe at a location on the reaction surface hybridizes with a corresponding type of target molecule to form a double-stranded structure, the presence of the corresponding target molecule in the test sample can be identified. When a plurality of nucleic acid probes corresponding to a plurality of types of target molecules are immobilized on the reaction surface, it is possible to identify the presence of a plurality of types of target molecules in the test sample, thereby achieving multiplex detection.

The first embodiment is a device embodiment corresponding to the present embodiment, and the present embodiment may be implemented in conjunction with the first embodiment. The relevant technical details mentioned in the first embodiment are still valid in the present embodiment, and are not repeated herein to minimize repetition. Accordingly, the relevant technical details mentioned in the present embodiment can also be applied in the first embodiment.

### Fifth Embodiment

A fifth embodiment of the present invention provides a detection method for a homogeneous multiplex detection device. FIG. 4D is a schematic diagram of a detection method for a homogeneous multiplex detection device of an embodiment of the present invention. The homogeneous multiplex detection device has been described in the first embodiment above, and as shown in FIG. 4D, the detection method may comprise:

forming closed circuits respectively by a plurality of nucleic acid probes immobilized on a reaction surface of a reaction chip and an electrical signal detector;

emitting excitation light by a light source and irradiating to the reaction surface of the reaction chip such that nucleic acid molecules formed by amplification of one or more target molecules within the reaction vessel can generate electrical signals after hybridization of the amplified target molecules with corresponding nucleic acid probe molecules;

detecting the electrical signals by an electrical signal detector;

identifying the type of one or more target molecules that hybridize with the corresponding nucleic acid probe molecules based on the types of the corresponding nucleic acid probes where the fluorescent signals are detected.

In one embodiment, the light source may be a laser light source, or a LED light source or other light sources. In one embodiment, an electrical signal may be generated by the photoelectric effect, wherein a photosensitizer realizing the photoelectric effect is introduced to the corresponding nucleic acid probe on a reaction chip by an amplified target molecule. When a target molecule hybridizes with a corresponding nucleic acid probe molecule to form a double-stranded molecule, a photosensitive molecular marker (such as a photosensitizer) is introduced. When the excitation light emitted by the light source irradiates the reaction surface of the reaction chip, the photosensitizer can generate charge separation. Since the charge separation can generate a potential difference or free electrons, the electrical signal can be detected by an electrical signal detector.

In one embodiment, the electrical signal detector may be a Field Effect Transistor to detect a potential difference; and in another embodiment, the electrical signal detector may be an electrochemical detection device to detect free electrons.

In one embodiment, the type of nucleic acid probe can be identified by the pre-defined position of the nucleic acid probe on the reaction surface of the reaction chip. As already described in the first embodiment above, when it is determined that a molecule of a known nucleic acid probe at a certain position on the reaction surface hybridizes with a corresponding type of a target molecule to form a double-stranded molecule, the presence of the target molecule in the test sample can be identified. When a plurality of types of nucleic acid probes corresponding to a plurality of types of target molecules are immobilized on the reaction surface, it is possible to identify the presence of a plurality of types of target molecules in the test sample, thereby realizing multiplex detection.

The first embodiment is a device embodiment corresponding to the present embodiment, and the present embodiment may be implemented in conjunction with the first embodiment. The relevant technical details mentioned in the first embodiment are still valid in the present embodiment, and thus are not repeated herein to minimize repetition. Accordingly, the relevant technical details mentioned in the present embodiment can also be applied in the first embodiment.

It is understood that the third embodiment of the present invention may be generally referred to as a fluorescent signal detection method, the fourth embodiment may be generally referred to as an optical signal detection method, and the fifth embodiment of the present invention may be generally referred to as an electrical signal detection method.

Although the present invention has been illustrated and described by reference to certain preferred embodiments, it should be understood by those of ordinary skill in the art that various changes may be made thereto in forms and details without departing from the principles and scopes of the present invention.

1. A homogeneous multiplex detection device, comprising:
a reaction vessel, providing a reaction space with two ends opposite to each other, wherein the first end of the reaction vessel is an open end and the second end of the reaction vessel is a closed-end, and being used for nucleic acid amplification reactions;
a reaction chip, positioning inside a reaction space and on the first end of the reaction vessel, and having a reaction surface where a plurality of types of nucleic acid probes corresponding to a plurality of types of target molecules are immobilized, wherein said nucleic acid probes are used for detecting nucleic acid molecules formed by amplifying the target molecules inside the reaction vessel;
a lid, detachably arranged on the first end of the reaction vessel to close said reaction space.

2. The device of claim 1, further comprises a first heater and a second heater, said first heater being arranged on the first end of said reaction vessel, and said second heater being arranged on the second end of said reaction vessel, so that thermal convection is formed when said reaction vessel is used for a nucleic acid amplification reaction.

3. The device of claim 1, wherein said reaction vessel is a tubular structure, and the first end and the second end of said reaction vessel are at the opposite end of each other in the lengthwise direction of said tubular structure, wherein said first end and said second end of said reaction vessel can be arranged concentrically or non-concentrically, and the cross-sections of said first end and said second end of said reaction vessel are the same or different.

4. The device of claim 3, wherein said cross-sections of said first end and said second end of said reaction vessel comprise at least one of a curved side and a straight side, respectively.

5. The device of claim 4, wherein said cross-sections of said first end and said second end of said reaction vessel have an inner diameter or a minimum side length from 0 0.5 mm to 5 mm, and said tubular structure has a length from 5 mm to 50 mm, and said reaction space has a volume from 5 μl to 5000 μl.

6. The device of claim 3, wherein said reaction chip and said lid are integrated together.

7. The device of claim 3, wherein the reaction surface of said reaction chip is oriented in a radial direction of said tubular structure or in a lengthwise direction of said tubular structure and towards said second end of said reaction vessel.

8. (canceled)

9. The device of claim 2, wherein said first heater is integrated into said reaction chip.

10. The device of claim 1, further comprises a signal detector, said signal detector detects a fluorescent signal, an optical signal or an electrical signal from a reaction chip.

11. The device of claim 1, wherein said plurality of types of target molecules includes one or more of RNA molecules or DNA molecules, RNA fragments in an RNA genome or DNA fragments in a DNA genome, and variant structures in RNA molecules or DNA molecules.

12. The device of claim 11, wherein said plurality of types of target molecules are originated from humans, animals, plants, microorganisms or are artificially or chemically synthesized, wherein said microorganisms include one or more of viruses, bacteria and fungi.

13. A method for operating a homogeneous multiplex detection device of claim 1, said method comprising:
adding a reagent and a test sample in the inside of a reaction space provided by a reaction vessel, wherein test sample comprising one or more target molecules to be detected;
heating the reaction vessel by the first heater and the second heater, so that the reagent and the test sample can form convection flow between the first end and the second end of the reaction vessel under the action of thermal convection, and the one or more target molecules to be detected in the test sample can hybridize not only with the corresponding primers in the reaction system to achieve amplification, but also with the complementary nucleic acid probe molecules immobilized on the reaction surface of the reaction chip.

14. The method of claim 13, wherein heating said first heater and heating said second heater are controlled separately so that the temperature on said first end of said reaction vessel is from 30° C. to 75° C. and the temperature on said second end of said reaction vessel is from

15. The method of claim 13, wherein said reagent includes primers and a DNA polymerase.

16. The method of claim 15, wherein said DNA polymerase has a 3′→5′ exonuclease activity.

17. A detection method for the homogeneous multiplex detection device according to claim 1, said method comprising:
emitting excitation light by a light source and irradiating a reaction surface of a reaction chip such that nucleic acid molecules produced by amplification of one or more target molecules in a reaction vessel can generate fluorescent signals after hybridization of the amplified target molecules with complementary nucleic acid probe molecules immobilized on said reaction surface of said reaction chip;
detecting said fluorescent signals by a fluorescent signal detector;
identifying the type of one or more target molecules that hybridize with the complementary nucleic acid probe molecules based on the positions or types of the corresponding nucleic acid probes where the fluorescent signals are detected.

18. The method of claim 17, wherein said fluorescent signals are generated by one or more of the methods of fluorescent dye direct excitation, dye intercalation, fluorescence resonance energy transfer, and fluorescence dequenching.

19. An detection method for the homogeneous multiplex detection device of claim 1, said method comprising:
forming closed circuits, respectively, between a plurality of nucleic acid probes immobilized on a reaction surface of a reaction chip and a power supply, such that nucleic acid molecules formed by amplification of one or more target molecules within a reaction vessel can generate light signals after hybridization of the amplified target molecules with complementary nucleic acid probe molecules;
detecting said light signals by a light signal detector;
identifying the type of one or more target molecules to be detected after hybridization of the amplified target molecules with said complementary nucleic acid probe molecules based on the light signals corresponding to the positions or the types of said nucleic acid probes.

20. An detection method for the homogeneous multiple detection device of claim 1, said method comprising:
forming closed circuits, respectively, between a plurality of nucleic acid probes immobilized on a reaction surface of a reaction chip and an electrical signal detector;
emitting excitation light by a light source and irradiating said reaction surface of said reaction chip such that nucleic acid molecules formed by amplification of one or more target molecules in a reaction vessel can generate electrical signals after hybridization of the amplified target molecules with the complementary nucleic acid probe molecules;
detecting said electrical signals by a electrical signal detector;
identifying the type of one or more target molecules that hybridize with said complementary nucleic acid probe molecules based on the positions or types of said complementary nucleic acid probes where said electrical signals are detected.

21. The method of claim 20, wherein said electrical signals are generated by means of a photoelectric effect, wherein the photosensitizer realizing the photoelectric effect is introduced into said corresponding nucleic acid probes by an amplified target molecules.
